(19) United States
(12) Patent Application Publication
(10) Pub. No.: US 2004/0151688 A1
(43) Pub. Date: Aug. 5, 2004

(54) Adhesive treatment for epistaxis

(75) Inventors: Anthony Sherbondy, Raleigh, NC (US); Gabriel N. Szabo, Raleigh, NC (US)

Correspondence Address: OLIFF & BERRIDGE, PLC, P.O. BOX 19928, ALEXANDRIA, VA, 22320, US

(73) Assignee: Closure Medical Corporation, Raleigh, NC (US)

(21) Appl. No.: 10/355,294

(22) Filed: Jan. 31, 2003

(51) Int. Cl. A61K 31/74

(52) U.S. Cl. 424/78.17

(57) ABSTRACT

A method of treating or preventing epistaxis includes applying a synthetic or semi-synthetic polymerizable monomer polymerizable monomer adhesive composition to a nasal area afflicted with or prone or susceptible to epistaxis, optionally with at least one of an additional anti-microbial or therapeutic agent, and allowing the polymerizable monomer composition to polymerize to form a polymer film over the nasal area.

### BACKGROUND OF THE INVENTION

1. Field of Invention

The present invention relates to treatment and preventative methods for epistaxis, or more commonly known as nosebleed. More particularly, the present invention relates to methods and compositions for the treatment or prevention of epistaxis.

2. Description of Related Art

Monomer and polymer adhesives are used in both industrial (including household) and medical applications. Included among these adhesives are the 1,1-disubstituted ethylene monomers and polymers, such as the α-cyanoacrylates. Since the discovery of the adhesive properties of such monomers and polymers, they have found wide use due to the speed with which they cure, the strength of the resulting bond formed, and their relative ease of use. These characteristics have made the α-cyanoacrylate adhesives the primary choice for numerous applications such as bonding plastics, rubbers, glass, metals, wood, and, more recently, biological tissues.

It is known that monomeric forms of α-cyanoacrylates are extremely reactive, polymerizing rapidly in the presence of even minute amounts of an initiator, including moisture present in the air or on moist surfaces such as animal (including human) tissue. Monomers of α-cyanoacrylates are anionically polymerizable or free radical polymerizable, or polymerizable by zwitterions or ion pairs to form polymers. Once polymerization has been initiated, the cure rate can be very rapid.

Medical applications of 1,1-disubstituted ethylene adhesive compositions include use as an alternate or an adjunct to surgical sutures and/or staples in wound closure, as well as for covering and protecting surface wounds such as lacerations, abrasions, bums, stomatitis, sores, minor cuts and scrapes, and other wounds. When an adhesive is applied to surfaces to be joined, it is usually applied in its monomeric form, and the resultant polymerization gives rise to the desired adhesive bond.

U.S. Pat. Nos. 5,514,371, 5,514,372, 5,575,997, 5,624,669, and 5,582,834 to Leung et al. disclose the addition of a therapeutic agent in a cyanoacrylate composition. The cyanoacrylate adhesive forms a matrix for the therapeutic agent, with the therapeutic agent being released in vivo over time from the matrix during biodegradation of the polymer.

U.S. Pat. No. 5,762,955 to Smith discloses a treatment for healthy, damaged, diseased, or infected biological tissue by applying a bioadhesive coating in conjunction with a medication. The treatment is directed, in part, to treating external biological tissue that may be affected by harmful afflictions such as bruises, burns, dermatological afflictions, infections, gashes, wounds, herpes sores, canker sores, or intra-oral lesions, and skin cancers such as leukemia. Smith further discloses several medications that may be used including corticosteroids, fluoroouracil, obtundants, anesthetics, antibiotics, fungicides, anti-inflammatory agents, antibacterial agents, antiseptic agents, and other medications or combinations of medications used in processes for healing tissue, promoting or preventing blood clotting, destroying cancer cells, palliative treatments and killing of bacteria or viruses.

U.S. Pat. No. 4,880,416 to Horiuchi et al., discloses a dermal bandage of a pre-formed film-like adhesive material for preventing dermally applied ointments, creams, solutions, powders, etc. from falling off, and for delivering drugs, such as anti-fungal agents, to affected parts of the skin. U.S. Pat. Nos. 5,716,607 and 5,716,608, both to Byram et al., disclose the use of cyanoacrylate adhesives to prevent ionization radiation damage to skin. Such damage is prevented by applying the cyanoacrylate polymer to the skin to be protected. U.S. Pat. No. 5,653,769 to Barley, Jr., et al., discloses protecting skin areas from irritation due to contact with artificial devices such as prosthetics, bandages and casts by applying a cyanoacrylate polymer to the desired skin areas that otherwise would be prone to ulceration or irritation by the devices.

U.S. Pat. No. 4,287,177 to Nakashima et al. discloses a protective covering material for forming a film or coat on the skin or wound surface, wherein the film may contain an anti-fungal agent that is controllably released when the composition is placed in contact with the skin. U.S. Pat. Nos. 5,684,042; 5,753,699; 5,762,919; 5,783,177; and 5,811,091 to Greff et al. disclose a cyanoacrylate composition with a compatible anti-fungal agent to form an anti-fungal polymeric cyanoacrylate film to be applied on mammalian skin as wound dressings, wound bandages, surgical incise drapes, wound closure materials and the like.

Epistaxis, more commonly referred to as “nosebleed,” is a hemorrhage in the nose causing bleeding. Epistaxis can range from minor to severe, where the bleeding can range from minor and for a short period of time to severe, persistent, and/or life-threatening in serious cases. Unless otherwise noted herein, “epistaxis” is used to generically refer to all such severities of the condition. Epistaxis can likewise be treated by methods ranging from simple at-home methods, such as by simple application of pressure to the nose, to more complicated methods requiring hospitalization and monitoring by a healthcare professional, such as by cautery or posterior and/or anterior nasal packing.

In prior studies, it was found that about 10-12 percent of adults report episodes of epistaxis each year, and about 60 percent of adults have at least one episode of epistaxis in their lifetime. Although many episodes are minor and self-limiting, i.e., the bleeding is short-termed and stops without hospitalization or medical treatment, there are over 800,000 reported visits to emergency departments in the United States each year for nosebleeds. This high frequency of epistaxis is related to the rich blood supply of the nasal septum and its vulnerability to external trauma. Most bleeding events originate from Little's area, which is located over the anterior-inferior portion of the nasal septum. In this area is a rich complex of vessels (Kisselbach's plexus) originating from the internal and external carotid arteries.

Currently, there are many methods to manage epistaxis including application of cautery, topical hemostatic agents, or nasal tampons (i.e., nasal packing). While usually effective, these methods can be associated with considerable adverse events such as infection, septal perforation, and aspiration. These methods, and their respective benefits and drawbacks, are described, for example, in L. H. Wurman et al., “The Management of Epistaxis,” Am. J. Otolaryngol., 1992:13, pp. 193-209, and G. D. Josephson, “Practical Management of Epistaxis,” Med. Clin. North Amer., 1991:75, p. 1311, the entire disclosures of which are incorporated herein by reference. This has led researchers and clinicians to continue to explore alternative methods for treating epistaxis.

In the past, fibrin glue has also found use, primarily in hospital settings, for treatment of epistaxis. The art has described that fibrin glue can be used as an alternative to packing or cautery to attempt to end the bleeding, by sealing the open vessels. Fibrin glue usage is disclosed, for example, in M. Vaiman et al., “Fibrin Glue Treatment for Epistaxis,” Rhinology J., Vol. 40, No. 2, pp. 88-91 (June 2002), and G. Porter, “Epistaxis,” Grand Rounds Presentation UTMB, Dept. of Otolaryngology (Apr. 10, 2002), the entire disclosures of which are incorporated herein by reference.

Other adhesive materials are known for use in and around the nasal passages, although not for treatment of epistaxis. For example, the literature has reported that cyanoacrylate adhesives can be used to remove foreign bodies lodged in a patient's nose, a condition that often occurs with small children. One such method is to apply cyanoacrylate glue to the end of a wooden or plastic applicator stick, press the applicator stick against the foreign body for about one minute to adhere to the foreign body, and then to remove the foreign body. See R. Cox, “Foreign Bodies, Nose,” from emedicine.com internet website, http://www.emedicine.com/emerg/topic186.htm (May 17, 2001), the entire disclosure of which is incorporated herein by reference. Similar procedures can be used to remove foreign bodies lodged in a patient's ear. See C. Stewart, “Foreign Body Removal,” EMR Textbook, available at http://www.thrombosis-consult.com/articles/textbook/131_foreignbody.htm (date unknown), the entire disclosure of which is incorporated herein by reference.

Despite the known uses of cyanoacrylate adhesives, such as described above, such adhesives have not been used in the treatment of epistaxis. Instead, the majority of epistaxis treatments include simple application of pressure to promote clotting, or the use of cautery or nasal packing. Therefore, there is a need for an alternative epistaxis treatment that quickly and reliable stops the bleeding.

### SUMMARY OF THE INVENTION

The present invention provides methods for treating or preventing epistaxis by applying a synthetic or semi-synthetic monomeric adhesive composition to the affected area. The synthetic or semi-synthetic monomeric adhesive composition of the present invention preferably comprises a polymerizable 1,1-disubstituted ethylene monomer such as a cyanoacrylate monomer, that may optionally include or be accompanied by an additional therapeutic agent. The composition, when polymerized, forms a film over the application site, but more importantly seals the open blood vessels to stop bleeding and promote clotting. The composition also assists to keep any active ingredients in contact with the application site for a longer time. The methods of the present invention thus provide fast and effective treatment or prevention of epistaxis, in a much less obtrusive manner as compared to the use of nasal packing or cautery.

The present invention provides an unexpected treatment and prevention composition and method for epistaxis, because polymerizable monomers such as 1,1-disubstituted ethylene monomers and cyanoacrylates have not previously been used to treat or prevent epistaxis, where there exists positive liquid (blood) flow in the confined spaces of the nasal passages. Moreover, while such polymerizable monomers have been variously used on other parts of the body, such as for sealing open wounds, their use for treating or preventing epistaxis is an entirely new and unexpected use of the materials.

The present treatment for epistaxis is advantageous for several reasons. First, because epistaxis represents a positive blood flow from the affected area, it is often difficult to keep therapeutic agents or fibrin glues in contact with the area for a sufficient amount of time to permit the agent or fibrin glue to achieve its desired function. The positive blood flow also poses difficulties in the use of nasal packing, as the nasal packing can become saturated with blood prior to the packing promoting clotting and stoppage of bleeding, and thus the packing may need to be replaced, at which time any partial clotting that may have occurred can be disrupted by the packing change. Second, because cautery and nasal packing are intrusive and, in some cases, painful, the methods of the present invention represent a less painful and less obtrusive means to treat epistaxis. The present invention addresses these drawbacks of the prior art, by providing a treatment method and composition that permit improved treatment of epistaxis.

Because cyanoacrylates tend to polymerize rapidly to form a relatively robust polymerized film, cyanoacrylates applied to an affected area of a patient's nasal passages can provide fast, effective protection over the affected area and other skin surfaces. The cyanoacrylate compound can also hold any optionally-applied active agents in place for a longer period of time and can accordingly significantly increase the time of exposure of an applied medication to the affected area, and ensure more effective treatment.

In addition to forming a stronger barrier at the application site while maintaining active agents in contact with the area, cyanoacrylate compounds are also desirable for the treatment of epistaxis because of their believed inherent anti-microbial properties. It has been demonstrated in at least some laboratory tests that some cyanoacrylate compounds or formulations provide microbial barrier and anti-microbial proliferation properties. Because of this anti-microbial property, such cyanoacrylate compounds and formulations may be especially desirable for treating epistaxis, even without the introduction or pre-application of other anti-microbial agents. Moreover, the anti-microbial properties of such cyanoacrylate compounds and formulations may also be beneficial in instances where an anti-microbial agent is applied before, or together with, the cyanoacrylate, because the cyanoacrylate can continue to provide its anti-microbial effect even after the applied anti-microbial agent has been completely absorbed or used up.

In addition, cyanoacrylate compounds are also useful as anti-microbial or therapeutic agent carriers or delivery agents. In this capacity, the cyanoacrylate compound provides the same protective, anti-microbial benefits, but also offers the added benefit of enhancing the treatment of the affected area by controllably releasing an anti-microbial or therapeutic agent to the affected area.

### DETAILED DESCRIPTION OF PREFERRED EMBODIMENTS

This invention is directed to methods of and compositions for treating or preventing epistaxis. In embodiments, the present invention is directed to methods comprising applying a synthetic or semi-synthetic monomeric adhesive composition, with or without optional additives, to an application site exhibiting, or prone to exhibit, epistaxis.

According to embodiments of the present invention, the adhesive composition can be applied alone, or it can be applied subsequent to or concurrent with the application of a separate therapeutic agent. Furthermore, in embodiments, the monomeric adhesive composition can itself include a therapeutic agent in addition to polymerizable monomer.

According to the present invention, “treat” (or other forms of the word such as “treating” and “treatment”) refers to employment of the methods and/or compositions against an active epistaxis condition, i.e., where positive blood flow exists. “Treat” thus encompasses both amelioration of effects of epistaxis, such as by reducing or preferably stopping blood flow out of the nose, and active reduction of epistaxis such as by promoting clotting at the affected area.

Treatment is thus distinguished from prevention, which involves areas that do not exhibit active epistaxis, but which may be prone to epistaxis, such as in individuals with a recurrent condition at the same site. According to the present invention, “prevent” (or other forms of the word such as “preventing” and “prevention”) refers to employment of the methods against a subsequent epistaxis event, such as at areas of the nose that are prone or susceptible to epistaxis. Accordingly, the present invention envisions prevention to be used by individuals or in cases where there is no current active bleeding, but where there is a reasonable probability that bleeding would occur while the polymerized film remains in place in the nose. Thus, for example, prevention could be used in individuals where a nosebleed has recently stopped, but it is believed that the nosebleed will otherwise resume. In other embodiments, prevention methods can be employed in individuals who suffer from certain congenital conditions that render the patient prone to nosebleeds. Other prevention methods are also within the scope of the present invention.

In embodiments of the invention, an adhesive composition can be used alone for treating or preventing epistaxis. In such embodiments, a patient or care-provider simply applies the composition to an area of the nose where the bleeding is occurring, or to an area where prevention of epistaxis is desired. The composition then is permitted to polymerize to form a robust polymeric coating that effectively covers and protects the application site. Anti-microbial properties of the composition may inhibit or kill microbes, and the robust polymeric coating that the composition forms protects the area and promotes bleeding stoppage and subsequent clotting.

Preferably, the composition is applied in a sufficient amount to entirely cover the desired area, which generally would correspond to an affected area where the bleeding is occurring, or to an area that is prone or susceptible to epistaxis. In embodiments, the composition covers an additional area around the desired area, for example to prevent blood from exiting around the edges of the applied material.

According to the present invention, the adhesive composition is preferably permitted to substantially or fully polymerize to form a polymer film before the treated area is permitted to contact other surfaces. Thus, for example, when the composition is applied within the nasal passages, any adjoining tissue surfaces are preferably kept separated from each other until the composition has substantially or fully polymerized, to prevent bonding the tissue areas together. Likewise, for example if the methods of the present invention are used in conjunction with nasal packing, the nasal packing is preferably not inserted until after the composition has substantially or fully polymerized, to prevent bonding the packing material to the tissue surfaces.

In further embodiments of the present invention, the adhesive composition can be applied over a medicament or other therapeutic agent. The medicament in this embodiment is not particularly limited, and can include any of the available medicaments for treating epistaxis, such as clotting agents or the like. The medicament can also be, or comprise, any suitable anti-microbial agent, as described below. In this embodiment, the medicament can be first applied to the affected or desired area, followed by application of a polymerizable adhesive composition. The medicament can be in any suitable form, including liquid, solid, powder, cream or the like, and can include only a medicament or can include other suitable additives such as diluents, carriers or the like. Where the medicament is in a liquid or a semi-liquid form, it is preferred that the medicament be permitted to dry, substantially or completely, prior to application of the adhesive composition. However, the adhesive composition can also be immediately applied over the applied medicament, or can be applied prior to drying of the medicament, if desired.

In embodiments of the present invention, an appropriate, preferably monomer-compatible, anti-microbial or therapeutic agent can be mixed with the polymerizable adhesive composition and a resultant composition applied to the affected or desired area. In this embodiment, the anti-microbial or therapeutic agent can be mixed with the polymerizable adhesive composition during manufacture (i.e. prior to packaging the materials), or immediately prior to use. However, the present invention is not limited to such embodiments. Thus, for example, the anti-microbial or therapeutic agent need not be monomer-compatible. In these embodiments, the anti-microbial or therapeutic agent can be mixed or combined with the polymerizable adhesive composition, usually just prior to application, and a resultant composition applied to the affected or desired area.

In further embodiments of the present invention the anti-microbial or therapeutic agent may also serve as a polymerization initiator or a stabilizer. Thus, the anti-microbial or therapeutic agent can provide not only a biological activity, but a chemical one as well.

Anti-microbial or therapeutic agents that also serve as polymerization initiators can initiate and/or accelerate the polymerization of the composition when applied to an affected or desired area of skin. Accelerated polymerization reduces the waiting time necessary after application, and makes the composition more convenient to apply. Suitable agents that can also serve as initiators include, but are not limited to, certain acidic and quaternary ammonium compounds. In embodiments where the agent also acts as a polymerization initiator or rate modifier, the present invention provides the additional advantage of not requiring that a further, separate polymerization initiator or rate modifier be used. Furthermore, in these embodiments, the agent is preferably located in a non-contacting relationship with the adhesive composition prior to use, so that premature polymerization of the adhesive composition does not occur.

Anti-microbial or therapeutic agents that also serve as stabilizers can extend the useful life of the composition. By increasing the useful life of the composition, the composition can be stored and packaged for longer periods of time without the risk of premature polymerization. Suitable anti-microbial or therapeutic agents that can also serve as stabilizers can include, but are not limited to, certain acidic and phenolic compounds. In embodiments where the agent also acts as a stabilizer for the adhesive composition, the present invention provides the additional advantage of not requiring that a further, separate stabilizer be used. Furthermore, in these embodiments, the agent is preferably located in a contacting relationship with the adhesive composition, such as being mixed with the adhesive composition, prior to use.

When the additives, such as an anti-microbial or therapeutic agent, are mixed with the synthetic or semi-synthetic monomer composition during storage, it is preferred that the mixture exhibit a sufficiently long shelf-life to permit economical commercial distribution of the mixture. Thus, for example, the mixture should exhibit a shelf-life, as measured at room temperature and moderate humidity (about 40% relative humidity), of at least about one year, and preferably at least about two or even at least about three years. Where the additive and monomer are not mixed during storage, it is still preferred that the separate components exhibit similar shelf-lives to those of a mixed composition. As used herein, “shelf-life” refers to the amount of time the container and composition therein can be held at approximately room temperature (21-25° C.) without degradation of the composition and/or container occurring to the extent that the composition and container cannot be used in the manner and for the purpose for which they were intended. Thus, while some degradation to either or both of the composition and container can occur, it must not be to such an extent that the composition and/or container is no longer useable. Shelf-life can thus be limited by physical or aesthetic changes to the containers or products contained therein, by chemical reactions occurring within the composition being stored, by chemical reactions between the container and the composition being stored, by degradation of the container itself, and the like.

Although a mixture of anti-microbial or therapeutic agent and polymerizable monomer, according to the present invention, is not limited to a specific ratio of agent to polymerizable monomer, the agent is preferably present in an effective amount, preferably a therapeutically effective amount for treating epistaxis or any of its related symptoms or side-effects.

When mixed or combined immediately prior to use, the anti-microbial or therapeutic agent can be mixed with the polymerizable monomer composition in a suitable container and thereafter applied. Alternatively, mixing can be conducted during the application process, for example by using an applicator that is loaded with the agent, which thereby mixes the agent with the adhesive composition during application.

Suitable anti-microbial agents include, but are not limited to, known agents such as parabens, cresols, azoles, allylamines, pollyenes, acidics, mercurials, quaternary ammonium compounds, other agents, non-polymer-stabilized compounds, i.e., that are not complexed with or otherwise part of a polymer species, mixtures thereof, and the like. Such anti-microbial agents should preferably be present in a therapeutically effective amount, particularly in cases where higher amounts may otherwise be toxic to the patient.

For example, suitable parabens include, but are not limited to, alkyl parabens and salts thereof, such as methylparaben, methylparaben sodium, ethylparaben, propylparaben, propylparaben sodium, butylparaben, and the like. Butyl-paraben is especially preferred as it can also act as a stabilizer for certain polymerizable monomers, such as cyanoacrylate monomers, in the adhesive composition. Suitable cresols include, but are not limited to, cresol, chlorocresol, and the like. Suitable azoles include, but are not limited to, voriconazole, ketoconazole, fluconazole, itraconazole, miconazole, clotrimazole, saperconazole, neticonazole, oxiconazole, isoconazole, sulconazole, tercanazole, tioconazole, and the like. Suitable allylamines include, but are not limited to, naftifine, SF86-327, and the like. Suitable polyenes include, but are not limited to, nyastatin, amphotericin B, pimaricin, and the like. Suitable acidics include, but are not limited to, benzoic acid and salts thereof, sorbic acid and salts thereof, propionic acids and salts thereof, boric acid and salts thereof, dehydroacetic acid, sulphurous and vanillic acids, and alkyl esters of pararhydrobenzoic acid. Suitable mercurials include but are not limited to, thiomersal, phenylmercuric acetate and nitrate, nitromersol and sodium ethylmercurithiosalicylate. Suitable quaternary ammonium compounds include, but are not limited to, benzalkonium chloride, cetylpyridinium chloride, benzethonium chloride, and cetyltrimethyl ammonium bromide. Other known agents that can be used include, but are not limited to, hydroquinone, pyrocatechol, resorcinol, 4-n-hexyl resorcinol, captan (i.e., 3a,4,7,7a-tetrahydro-2-((trichloromethyl)thio)-1H-isoindole-1,3(2H)-dione), benzalkonium chloride, benzalkonium chloride solution, benzethonium chloride, benzoic acid, benzyl alcohol, cetylpyridinium chloride, chlorobutanol, dehydroacetic acid, o-phenylphenol, phenol, phenylethyl alcohol, potassium benzoate, potassium sorbate, sodium benzoate, sodium dehydroacetate, sodium propionate, sorbic acid, thimerosal, thymol, chlorothymol, alcohols, chlorobutanol, phenoxy-2-ethanol, benzyl alcohol, P-phenylethyl alcohol, chlorhexidine, 6-acetoxy-2,4-dimethyl-m-dioxane 2, 4,4′ trichloro-2′-hydroxy-diphenylether, imidizoldinylether urea compound, bromo-2-nitropropanediol-1,3 5-bromo-5-nitrol-1,3 dioxane 2-methyl 1-4-isothiazolin-3-one and 5 chloro derivative 1-(3-chloroallyl)-3,5,7-triazo 1-azoniaadamantane chloride (Dowicil 200), phenylmercuric compounds such as phenylmercuric borate, phenylmercuric nitrate and phenylmercuric acetate, formaldehyde, formaldehyde generators such as the Germall II® preservative and Germall 115® preservative (imidazolidinyl urea, available from Sutton Laboratories, Charthan, N.J.), morpholines, salicylic and benzoic acids, sodium and potassium iodides, flucytosine, 5-flucytosine, griseofulvin, terbinafine, cidofovir, famicoclovir, valacyclovir, echinocandins, pneumocandins, pradimicins, benanomicins, nikkomycins, amorolfine, polyoxins, duanorubicin citrate, doxorubicin hydrochloride, tolnaftate, ciclopirox, butenafine, and ergestrol biosynthesis inhibitors.

Non-polymer-stabilized compounds, i.e., that are not complexed with or otherwise part of a polymer species, which can be either soluble or insoluble in the monomeric composition, can also be used. Where the compounds are insoluble in the monomeric composition, they must generally be capable of releasing species, such as ions, which are soluble in the monomer composition and provide the anti-microbial effect. Thus such compounds either themselves are, or provide, the anti-microbial agent. Such suitable non-polymer-complexed materials include, but are not limited to, metals and metal compounds. Examples of such metal compounds or elemental metals include, but not limited to, mercurial compounds, such as phenolmercuric chloride, phenolmercuric acetate, acetomeroctol, nitromersol, thimerosal, mercurochrome, mercuric chloride, and mercuric iodide; elemental metals, such as silver and copper; and metal compounds, such as copper chloride, copper sulfate, copper peptides, zinc chloride, zinc sulfate, zinc salts of cyanoacrylic acid, zinc salts of cyanoacetic acid, zinc salts of dicyanoglutaric acid, zinc salts of rosin, zinc oxide, zinc salts of polycyanoacrylic acid, zinc salts of polyacrylic acid, zinc bacitracin, zinc salicylate, zinc stearate, zinc citrate, zinc lactate, silver nitrate, silver iodide, silver acetate, silver benzoate, silver carbonate, silver chloride, silver citrate, silver oxide, silver sulfate, and tincture of iodine; as well as mixtures thereof and the like. Copper peptides are discussed, for example, in “Copper: An Essential Element for Life,” ProCyte Corporation, available on the world wide web at http://www.humatech.com/technology.html (Oct. 28, 1999), the entire disclosure of which is incorporated herein by reference. Further information on anti-microbial activities of metals can be found, for example, in S. Seymour Block, Disinfection, Sterilization and Preservation, 3^{rd} Ed., Philadelphia:Lea & Febiger, 1983, the entire disclosure of which is incorporated herein by reference. The ions from the metal, which constitute the anti-microbial agent, diffuse into and through the adhesive composition.

Other suitable anti-microbial agents include the various compounds identified as such in The Merck Index, 12^{th} Ed. (1996), incorporated herein by reference. Such agents include acrisorein, 3-amino-4-hydroxybutyric acid, ammonium mercuric chloride, amorolfine, amphotericin B, anthralin, azaserine, benzoic acid, bifonazole, biphenamine, boric acid, bromosalicylchloranilide, buclosamide, butenafine, butoconazole, calcium propionate, candicidin, chlordantoin, chlormidazole, chlorphenesin, chlorquinaldol, ciclopirox, cloconazole, clotrimazole, cloxyquin, coparaffinate, m-cresyl acetate, cupric sulfate, dermostatin, diamthazole dihydrochloride, econazole, enilconazole, etisazol, exalamide, fenticonazole, filipin, fluconazole, flucytosine, flutrimazole, fungichromin, griseofulvin, hachimycin, halethazole, hamycin, hexetidine, isoconazole, itraconazole, ketoconazole, lanoconazole, loflucarban, lucensomycin, Magenta I, mepartricin, 2-(methoxymethyl)-5-nitrofuran, miconazole, monensin, myxin, naftifine, natamycin, neomycin undecylenate, nifuratel, nystatin, oligomycins, omoconazole, ontianil, oxiconazole nitrate, pecilocin, perimycin, phenylmurcuric nitrate (basic), potassium iodide, propionic acid, pyrithione, pyrrolnitrin, rubijervine, salicylanilide, salicylic acid, saperconazole, sertaconzole, siccanin, sodium propionate, sulbentine, sulconazole, tenonitrozole, terbinafine, terconazole, thimerosal, tioconazole, tolciclate, tolindate, tolnaftate, triacetin, 2,4,6-tribromo-m-cresol, tubercidin, ujothion, undecylenic acid, viridin, zinc propionate, mixtures thereof, and the like.

In other embodiments of the present invention, one or more vasoconstrictor agents can also be utilized. When so utilized, the vasoconstrictor agent can be incorporated directly into the polymerizable monomer composition, or it can be included separate from the polymerizable monomer composition, such as in a separate container in a kit, in a separate compartment of an applicator, or the like. Suitable vasoconstrictor agents include, but are not limited to, herbs and herb products that have vasoconstrictor properties, including but not limited to Aesculus hippocastanum (Hippocastanaceac), Corylus avellana (Betulaceae), Ephedra sinica (Ma Huang), Hamamelis virginiana (Witch Hazel), Hydrastis canadensis (Goldenseal), Lycopus virginicus (Bugleweed), Aspidosperma quebracho (Quebracho blanco), Cupressus sempervirens (Cupressaceae), Cytisus scoparius (Fabaceac), Gossypium arboreum (Malvaceae), Gossypium herbaceum (Malvaceae), Hedera helix (Araliaceac), Phellodendron amurense (Rutaceae), Plectranthus mollis (Lamiaceae), Polygonum hydropiper (Polygonaceae), Seseli sibiricum (Apiaceae), Strychnos ignatius (Loganiaceae), Strychnos nux-vomica (Loganiaceac), Urtica dioica (Urticaceae), and mixtures thereof. Other suitable vasoconstrictors include, but are not limited to, phenylephrine hydrochloride, etilefrine hydrochloride, acetylcholine, bradykinin, naphazoline hydrochloride, Angiotensin II (AII), epinephrine, cocaine, lidocaine, anesthetics, mixtures thereof, and the like. Mixtures of two or more vasoconstrictors can also be used, as desired. The vasoconstrictor can be included in any suitable and effective amount for its intended purpose.

In addition, in embodiments where monomer additives including, but not limited to those listed above, are insoluble with the monomer composition and/or that would cause premature polymerization of the monomer, the additive can be applied to the application site before applying the monomer composition. In such embodiments, the additive and the monomer composition can be provided, for example, in separate packages in a kit.

In other embodiments, where such additives are soluble with the monomer composition and/or would not cause premature polymerization of the monomer, the additives can be combined with the monomer composition during manufacture of the composition. Moreover, in cases where the additive is soluble with the monomer composition, the additive can be applied before the monomer composition is applied, it can be pre-mixed with and applied together with the monomer composition, it can be mixed together with the monomer composition immediately before application, or it can even be applied after the monomer composition has been applied. As a result, in cases where a soluble additive is to be applied, the additive and the composition can be provided in a kit where the additive and the monomer composition are pre-mixed, or the additive and the monomer composition can be provided separately to be applied separately or mixed together immediately prior to, during, or after application.

Although a mixture of additive and monomer composition according to the present invention is not limited to a specific ratio of additive to polymerizable monomer, the additive is preferably present in an effective amount, and preferably in a therapeutically effective amount.

When mixed immediately prior to use, the additive can be mixed with the polymerizable monomer composition in a suitable container and thereafter applied. Alternatively, mixing can be conducted during the application process, for example by using an applicator loaded with the additive, which thereby mixes the additive with the adhesive composition during application.

In addition, as discussed above with respect to suitable anti-microbial and therapeutic agents, other additives may also serve as polymerization initiators or rate modifiers. Also, any other additives may serve as stabilizers for the adhesive composition.

In embodiments, the monomer composition and/or its packaging can be sterilized. However, sterilization is not required, particularly in view of the fact that the composition will be used in the nasal passages. Furthermore, whether or not the composition and container is sterilized, the composition can further include one or more suitable preservative, as described below.

Sterilization of the monomer composition and/or its packaging can be accomplished by techniques known to the skilled artisan, and is preferably accomplished by methods including, but not limited to, chemical, physical, and/or irradiation methods. Examples of chemical methods include, but are not limited to, exposure to ethylene oxide or hydrogen peroxide vapor. Examples of physical methods include, but are not limited to, sterilization by heat (dry or moist) or retort canning. Examples of irradiation methods include, but are not limited to, gamma irradiation, electron beam irradiation, and microwave irradiation. A preferred method is electron beam irradiation, as described in U.S. Pat. No. 6,143,805, the entire disclosure of which is incorporated herein by reference. The composition should also show low levels of toxicity to living tissue during its useful life. In preferred embodiments of the present invention, the composition is sterilized to provide a Sterility Assurance Level (SAL) of at least 10⁻³. In embodiments, the Sterility Assurance Level may be at least 10⁻⁴, or may be at least 10⁻⁵, or may be at least 10⁻⁶.

The monomer (including prepolymeric) adhesive composition may include one or more polymerizable monomers, which preferably are synthetic or semi-synthetic monomers. Preferred monomers that may be used in this invention are readily polymerizable, e.g. anionically polymerizable or free radical polymerizable, or polymerizable by zwitterions or ion pairs to form polymers. Such monomers include those that form polymers, that may, but do not need to, biodegrade. Such monomers are disclosed in, for example, U.S. Pat. Nos. 5,328,687, 5,928,611 and 6,183,593, U.S. patent application Ser. No. 09/430,177, filed on Oct. 29, 1999, and U.S. Pat. No. 6,183,593, which are hereby incorporated in their entirety by reference herein.

Preferred monomers include 1,1-disubstituted ethylene monomers, such as α-cyanoacrylates including, but not limited to, alkyl α-cyanoacrylates having an alkyl chain length of from about 1 to about 20 carbon atoms or more, preferably from about 3 to about 8 carbon atoms.

The α-cyanoacrylates of the present invention can be prepared according to several methods known in the art. U.S. Pat. Nos. 2,721,858, 3,254,111, 3,995,641, and 4,364,876, each of which is hereby incorporated in its entirety by reference herein, disclose methods for preparing α-cyanoacrylates.

Preferred α-cyanoacrylate monomers used in this invention include methyl cyanoacrylate, ethyl cyanoacrylate, n-butyl cyanoacrylate, 2-octyl cyanoacrylate, methoxyethyl cyanoacrylate, ethoxyethyl cyanoacrylate, dodecyl cyanoacrylate, 2-ethylhexyl cyanoacrylate, butyl cyanoacrylate, 3-methoxybutyl cyanoacrylate, 2-butoxyethyl cyanoacrylate, 2-isopropoxyethyl cyanoacrylate, 1-methoxy-2-propyl cyanoacrylate, hexyl cyanoacrylate, or dodecylcyanoacrylate.

Other suitable cyanoacrylates for use in the present invention also include, but are not limited to, alkyl ester cyanoacrylate monomers such as those having the formula

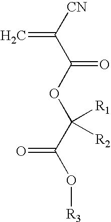

wherein R₁ and R₂ are, independently H, a straight, branched or cyclic alkyl, or are combined together in a cyclic alkyl group, and R₃ is a straight, branched or cyclic alkyl group. Preferably, R₁ is H or a C₁, C₂ or C₃ alkyl group, such as methyl or ethyl; R₂ is H or a C₁, C₂ or C₃ alkyl group, such as methyl or ethyl; and R₃ is a C₁-C₁₆ alkyl group, more preferably a C₁-C₁₀ alkyl group, such as methyl, ethyl, propyl, butyl, pentyl, hexyl, heptyl, octyl, nonyl or decyl, and even more preferably a C₂, C₃ or C₄ alkyl group. Such alkyl ester cyanoacrylates and other suitable monomers are disclosed in, for example, U.S. patent applications Ser. Nos. 09/630,437, filed Aug. 2, 2000, and 09/919,877, filed Aug. 2, 2001, the entire disclosures of which are incorporated herein by reference.

Examples of preferred alkyl ester cyanoacrylates include, but are not limited to, butyl lactoyl cyanoacrylate (BLCA), butyl glycoloyl cyanoacrylate (BGCA), ethyl lactoyl cyanoacrylate (ELCA), and ethyl glycoloyl cyanoacrylate (EGCA). BLCA may be represented by the above formula, wherein R₁ is H, R₂ is methyl and R₃ is butyl. BGCA may be represented by the above formula, wherein R₁ is H, R₂ is H and R₃ is butyl. ELCA may be represented by the above formula, wherein R₁ is H, R₂ is methyl and R₃ is ethyl. EGCA may be represented by the above formula, wherein R₁ is H, R₂ is H and R₃ is ethyl.

The composition may optionally also include at least one other plasticizing agent that assists in imparting flexibility to the polymer formed from the monomer. The plasticizing agent preferably contains little or no moisture and should not significantly affect the stability or polymerization of the monomer. Examples of suitable plasticizers include but are not limited to tributyl citrate, acetyl tri-n-butyl citrate (ATBC), polymethylmethacrylate, polydimethylsiloxane, hexadimethylsilazane and others as listed in U.S. Pat. No. 6,183,593, the disclosure of which is incorporated in its entirety by reference herein.

The composition may also optionally include at least one thixotropic agent. Suitable thixotropic agents are known to the skilled artisan and include, but are not limited to, silica gels such as those treated with a silyl isocyanate, and optionally surface treated titanium dioxide. Examples of suitable thixotropic agents and thickeners are disclosed in, for example, U.S. Pat. No. 4,720,513, and U.S. Pat. No. 6,310,166, the disclosures of which are hereby incorporated in their entireties by reference herein.

The composition may optionally also include thickeners. Suitable thickeners may include poly (2-ethylhexy methacrylate), poly(2-ethylhexyl acrylate) and others as listed in U.S. patent application Ser. No. 09/472,392 filed Dec. 23, 1999, the disclosure of which is incorporated by reference herein in its entirety.

The composition may also optionally include at least one natural or synthetic rubber to impart impact resistance. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. Nos. 4,313,865 and 4,560,723, the disclosures of which are hereby incorporated in their entireties by reference herein.

The composition may optionally also include one or more stabilizers, preferably both at least one anionic vapor phase stabilizer and at least one anionic liquid phase stabilizer. These stabilizing agents may inhibit premature polymerization. Suitable stabilizers may include those listed in U.S. Pat. No. 6,183,593, the disclosure of which is incorporated by reference herein in its entirety. Furthermore, certain stabilizers may also function as anti-microbial agents, such as, for example, various acidic anti-microbials, as identified above.

The stability, and thus the shelf-life, of some monomeric adhesive compositions can be further enhanced and extended through careful regulation of the packaging. Treated (e.g., fluorinated polymer) packaging such as that disclosed in copending U.S. patent application Ser. No. 09/430,289, filed Oct. 29, 1999, which is hereby incorporated by reference herein in its entirety, is preferred and may reduce the amount of stabilizer that is combined into the composition. As mentioned above, certain stabilizers including, but not limited to, certain acidics can also function as anti-microbial agents. In this case, the amount of the anti-microbial/stabilizer material is either not reduced below a level to provide the desired anti-microbial effect, or a further anti-microbial/non-stabilizing agent is added to ensure that the desired anti-microbial effect is provided.

The compositions may also include pH modifiers to control the rate of degradation of the resulting polymer, as disclosed in U.S. Pat. No. 6,143,352, the entire disclosure of which is hereby incorporated by reference herein in its entirety.

To improve the cohesive strength of adhesives formed from the compositions of this invention, difunctional monomeric cross-linking agents may be added to the monomer compositions of this invention. Such crosslinking agents are known. U.S. Pat. No. 3,940,362 to Overhults, which is hereby incorporated herein in its entirety by reference, discloses exemplary cross-linking agents.

The compositions of this invention may further contain fibrous reinforcement and colorants such as dyes, pigments, and pigment dyes. Examples of suitable fibrous reinforcement include PGA microfibrils, collagen microfibrils, and others as described in U.S. Pat. No. 6,183,593, the disclosure of which is incorporated by reference herein in its entirety.

The polymerizable compositions useful in the present invention may also further contain one or more preservatives, for prolonging the storage life of the composition. Suitable preservatives, and methods for selecting them and incorporating them into adhesive compositions, are disclosed in U.S. patent application Ser. No. 09/430,180, the entire disclosure of which is incorporated herein by reference. Such preservatives can be in addition to any anti-microbial agent that may or may not be added to the composition, as described above. Such preservatives can be included irrespective of whether the composition and containers are sterilized.

In embodiments of the present invention, the composition and/or its applicator may contain materials such as a polymerization initiator, accelerator, rate-modifier, and/or cross-linking agent for initiating polymerization and/or cross-linking of the polymerizable monomer material. Suitable materials and applicators and packaging systems are disclosed in U.S. Pat. No. 5,928,611 and U.S. patent applications Ser. Nos. 09/430,177, 09/430,176, 09/430,289, 09/430,290, and 09/430,180 filed Oct. 29, 1999; 09/343,914 filed Jun. 30, 1999; 09/385,030 filed Aug. 30, 1999; and 09/176,889 filed Oct. 22, 1998; the entire disclosures of which are incorporated herein by reference.

In addition to the various advantages identified above, the methods of the present invention provide additional advantages over state-of-the art epistaxis treatment protocols. For example, an advantage of using polymerizable monomeric adhesive compositions such as cyanoacrylate to treat epistaxis is that the compositions polymerize on contact with the injured mucosa, forming an occlusive dressing and creating a moist wound environment conducive to healing. Healing of such injured mucosa is thus expected to be quicker and more natural than for other treatment protocols, such as nasal packing and cautery. Furthermore, this occlusive coating protects the mucosa from further injury while also functioning as a microbial barrier.

Still further, the present invention provides treatment protocols that are considered advantageous both to the healthcare professional and the patient. It is believed that in the clinical scenario, use of topically applied polymerizable monomeric adhesive compositions such as cyanoacrylate would generally not require any follow-up visits by the patient, which are often required with many of the other therapeutic methods.

Although it has been known to use non-synthetic materials, such as fibrin glue, for treating epistaxis in the past, the present inventors have discovered that the use of synthetic or semi-synthetic monomer materials provide significant advantages over the prior art use of fibrin glue. For example, the nature of the monomer species as being synthetic or semi-synthetic versus non-synthetic or natural provides advantages in wider permitted use of the product. Furthermore, the synthetic or semi-synthetic monomers composition of the present invention are generally cheaper to produce and use as compared to non-synthetic products such as fibrin glue, while providing a stronger film upon setting. Where setting time is important, as in the methods of the present invention, the synthetic or semi-synthetic monomer compositions also provide an improved or faster set time, allowing for a shorter treatment time. Likewise, the resultant film according to the present invention, in addition to being stronger, provides a better barrier to prevent bacteria from entering the wound site, while also in embodiments providing anti-microbial properties. None of these benefits were previously provided by glue product treatment methods for epistaxis, such as fibrin glue treatments.

According to the present invention, any suitable applicator can be used to apply the composition to the affected areas of skin. Suitable applicators and packaging systems are disclosed in, for example, U.S. Pat. Nos. 5,928,611 and 6,352,704 and U.S. patent applications Ser. Nos. 09/430,177, 09/430,176, 09/430,289, 09/430,290, and 09/430,180 filed Oct. 29, 1999; 09/385,030 filed Aug. 30, 1999; 09/176,889 filed Oct. 22, 1998, and 09/898,006 filed Jul. 5, 2001; the entire disclosures of which are incorporated herein by reference.

### EXAMPLES

### Example 1

Methods

A prospective longitudinal interventional experiment is conducted. Seven female pigs are used in this study. Domestic pigs are chosen as the experimental animals because their nasal blood flow most closely resembles that of humans. Animals are given a standard diet ad lib several days prior to the investigation and are fasted overnight before any procedures. Housing and care for animals is in accordance with the National Research Council guidelines.

Study Protocol and Interventions

All animals are sedated with Talazine® (Tiletamine and Zolazepam, Fort Dodge Lab, Fort Dodge, Iowa) 5 mg/kg IM. The pigs are then intubated endotracheally and maintained under a surgical plane of anesthesia with isoflurane 0.5 2.5% in room air.

Choice of Wounding Instrument

During the first phase of the experiment a variety of instruments are used to create wounds on the nasal mucosa of one of the animals. Instruments that are tested include a variety of metal brushes (usually intended for cleaning test tubes), rotating metal discs and brushes that are operated using a Dremel® drill (Dremel, Racine, Wis.), and 4-mm surgical punches (Miltex Instrument Company Inc., Lake Success, N.Y.). Only the surgical punches result in consistent and uniform wounds that result in bleeding for at least 4 minutes in the unheparinized animal. The animal is then heparinized with intravenous heparin 20 units/kg and additional wounds are created with the surgical punch. These wounds consistently result in prolonged bleeding that lasts at least 20 minutes.

Creation of Wounds

For the remainder of the study, all of the wounds are created using a 4 mm surgical punch biopsy. The surgical punch is applied to the nasal mucosa over the anterior inferior nasal septum using slight pressure only. The punch is then rotated 90° clockwise, 90° counterclockwise, 90° clockwise, and finally 90° counterclockwise. This results in bleeding within 10-20 seconds of injury in all cases.

A standardized full thickness mucosal wound is created using the above procedure over the anterior-inferior portion of the nasal septae of each of the 7 animals using the surgical punch. The time to hemostasis is determined for this non treated control wound. A second wound is then created and allowed to bleed for a period of 30 seconds after which it is treated with the topical cyanoacrylate formulation, as described below. The animals are then fully heparinized and two additional wounds are created in each animal, one of which is immediately treated with cyanoacrylate while the other is allowed to bleed for 10 minutes and then treated with cyanoacrylate The animals are allowed to recover from anesthesia and the wounds are observed for 24 hours to determine the rates of re bleeding, if any.

Application of Cyanoacrylate Adhesive

A stabilized 2-octyl cyanoacrylate adhesive composition is commercially available in a liquid (monomeric) form in a plastic bottle together with a double-ended swab (Colgate® ORABASE® Soothe-N-Seal® product, available from Colgate Palmolive, El Paso, Tex.). Both ends of the swab are tipped in foam of differing shapes: one is a rounded paddle used for blotting the wound dry and the other is pointed for application of the cyanoacrylate. After removing the bottle cap, the pointed end of the swab is dipped into the bottle until all the liquid formulation (0.16 ml) is soaked up. The wound is then dried by gently dabbing the round paddle end of the swab against it. The swab is then whirled 180 degrees in the hand of the investigator and the pointed end is used to dab the wound with OCA for one second. If hemostasis is not achieved, the wound is again dabbed dry with the rounded end of the swab and the cyanoacrylate is applied a second time. If hemostasis is not achieved, additional applicators are used in succession as previously described until hemostasis is complete.

Results and Discussion

The control group consists of wounds that are not treated with cyanoacrylate and those that are created after full heparnization and before cyanoacrylate application. The treatment group consists of all wounds that are not treated with cyanoacrylate. Data analysis is conducted using SPSS 11.0 for Windows (SPSS, Inc., Chicago, Ill.). The two groups of wounds are compared with t-tests and χ² tests for continuous and dichotomous variables respectively. A sample size of 12 wounds in each group (control vs. cyanoacrylate) has 95% power to detect a 50% difference in the rates of complete hemostasis.

During the second phase of the study, six pigs are used. In each pig a total of four wounds are created: two on either side of the nasal septum. Thus, the total number of punch wounds created is 24. 12 wounds are created before heparinization while 12 wounds are created after full heparinization. A relatively small 4 mm sized surgical punch is used to create the wounds. This allows creation of four wounds on each animal reducing the total number of animals required for the study. Furthermore, creation of wounds both before and after full heparinization allows evaluation of the hemostatic properties of the therapeutic agent in a normal as well as coagulopathic animal.

The mean time from injury to hemostasis in control wounds is 4 minutes, 19 second (SD=23 seconds). Complete and sustained hemostasis is achieved in all wounds treated with the cyanoacrylate. The mean time from injury to hemostasis in the group of wounds treated with OCA is 123 seconds (SD=112) and 101 seconds (SD=117) with and without prior heparinization. The time to hemostasis is significantly shorter in the wounds treated with cyanoacrylate vs. those left to clot on their own (mean difference 150 seconds, 95% CI, 92 to 209 seconds, P<0.001). However, the difference between wounds treated with cyanoacrylate with or without prior heparinization is not statistically different (mean difference 22 seconds, 95% CI, 101 to 145 seconds, P=0.21). One third of the non heparinized wounds are treated with one cyanoacrylate applicator while one half of the heparinized wounds stop bleeding after one application of cyanoacrylate (Table, P=0.57). In many wounds, hemostasis is obtained immediately after applying the cyanoacrylate to the wound. None of the wounds treated with cyanoacrylate require more than 3 applications. Bleeding does not recur in any of the wounds over a 24-hr observation period.

TABLE
Number of Cyanoacrylate Applicators Required to
Achieve Hemostasis

| | Unheparinized Wounds | Heparinized Wounds |
|---|---|---|
| | (n = 6) | (n = 12) |
| 1 applicator | 2 (33%) | 6 (50%) |
| 2 applicators | 3 (50%) | 3 (25%) |
| 3 applicators | 1 (17%) | 3 (25%) |

Conclusion

The results of the study indicate that the cyanoacrylate composition is highly effective as a hemostatic agent as evidenced by rapid and complete hemostasis in all treated wounds even after full heparinization.

While the invention has been described with reference to preferred embodiments, the invention is not limited to the specific examples given, and other embodiments and modifications can be made by those skilled in the art without departing from the spirit and scope of the invention.

What is claimed is:

1. A method of treating or preventing epistaxis, comprising:
a) applying an adhesive composition comprising a synthetic or semi-synthetic polymerizable monomer to a nasal area that is afflicted with or susceptible to epistaxis; and
b) allowing said synthetic or semi-synthetic polymerizable monomer to polymerize to form a polymer film over said nasal area.

2. The method of claim 1, wherein said method is for treating said epistaxis, and said nasal area is afflicted with said epistaxis.

3. The method of claim 1, wherein said method is for preventing said epistaxis, and said nasal area is susceptible to said epistaxis.

4. The method of claim 1, wherein said synthetic or semi-synthetic polymerizable monomer comprises a 1,1-disubstituted ethylene monomer.

5. The method of claim 1, wherein said synthetic or semi-synthetic polymerizable monomer is an α-cyanoacrylate monomer.

6. The method of claim 1, wherein said synthetic or semi-synthetic polymerizable monomer comprises at least one member selected form the group consisting of ethyl cyanoacrylate, butyl cyanoacrylate, and 2-octyl cyanoacrylate.

7. The method of claim 1, further comprising combining at least one of an anti-microbial or therapeutic agent with the polymerizable monomer on the nasal area so that the at least one agent serves as a polymerization initiator for said polymerizable monomer composition.

8. The method of claim 1, wherein said composition further comprises at least one stabilizing agent for said polymerizable monomer.

9. The method of claim 8, wherein said stabilizing agent is also at least one of an anti-microbial agent or a therapeutic agent.

10. The method of claim 1, wherein said composition comprises at least one plasticizer.

11. The method of claim 10, wherein the plasticizer is selected from the group consisting of tributyl citrate, acetyl tributyl citrate, polymethylmethacrylate, polydimethylsiloxane and hexadimethylsilazane.

12. The method of claim 1, wherein the composition further comprises at least one of an anti-microbial or therapeutic agent.

13. The method of claim 12, wherein said method is for treating said epistaxis, and said nasal area is afflicted with epistaxis.

14. The method of claim 12, wherein said method is for preventing said epistaxis, and said nasal area is susceptible to said epistaxis.

15. The method of claim 12, wherein the composition further comprises said at least one anti-microbial agent and said at least one anti-microbial agent is selected from the group consisting of parabens, cresols, azoles, allylamines, pollyenes, acidics, mercurials, quaternary ammonium compounds, and non-polymer-stabilized compounds.

16. The method of claim 12, wherein the composition further comprises said at least one anti-microbial agent and said at least one anti-microbial agent is a paraben selected from the group consisting of alkyl parabens having an alkyl group of from 1-4 carbon atoms.

17. The method of claim 12, wherein the composition further comprises said at least one anti-microbial agent and said at least one anti-microbial agent is selected from the group consisting of methylparaben, methylparaben sodium, ethylparaben, propylparaben, propylparaben sodium, butylparaben, cresol, chlorocresol, voriconazole, ketoconazole, fluconazole, itraconazole, miconazole, clotrimazole, saperconazole, neticonazole, oxiconazole, isoconazole, sulconazole, tercanazole, tioconazole, naftifine, SF86-327, nyastatin, amphotericin B, pimaricin, benzoic acid and salts thereof, sorbic acid and salts thereof, propionic acids and salts thereof, boric acid and salts thereof, dehydroacetic acid, sulphurous and vanillic acids, alkyl esters of pararhydrobenzoic acid, thiomersal, phenylmercuric borate, phenylmercuric acetate and phenylmercuric nitrate, nitromersol, sodium ethylmercurithiosalicylate, benzalkonium chloride, cetylpyridinium chloride, benzethonium chloride, cetyltrimethyl ammonium bromide, hydroquinone, pyrocatechol, resorcinol, 4-n-hexyl resorcinol, 3a,4,7,7a-tetrahydro-2-((trichloromethyl)thio)-1H-isoindole-1,3(2H)-dione, benzalkonium chloride, benzethonium chloride, benzoic acid, benzyl alcohol, cetylpyridinium chloride, chlorobutanol, dehydroacetic acid, o-phenylphenol, phenol, phenylethyl alcohol, potassium benzoate, potassium sorbate, sodium benzoate, sodium dehydroacetate, sodium propionate, sorbic acid, thimerosal, thymol, chlorothymol, alcohols, chlorobutanol, phenoxy-2-ethanol, benzyl alcohol, P-phenylethyl alcohol, chlorhexidine, 6-acetoxy-2,4-dimethyl-m-dioxane 2,4,4′trichloro-2′-hydroxy-diphenylether, imidizoldinylether urea compound, bromo-2-nitropropanediol-1,3 5-bromo-5-nitrol-1,3 dioxane, 2-methyl 1-4-isothiazolin-3-one and 5 chloro derivative, 1-(3-chloroallyl)-3,5,7-triazo 1-azoniaadamantane chloride, formaldehyde, imidazolidinyl urea, morpholines, salicylic acids, benzoic acids, sodium iodides and potassium iodides, flucytosine, 5-flucytosine, griseofulvin, terbinafine, cidofovir, famicoclovir, valacyclovir, echinocandins, pneumocandins, pradimicins, benanomicins, nikkomycins, amorolfine, polyoxins, duanorubicin citrate, doxorubicin hydrochloride, tolnaftate, ciclopirox, butenafine, ergestrol biosynthesis inhibitors, acrisorein, 3-amino-4-hydroxybutyric acid, ammonium mercuric chloride, amorolfine, anthralin, azaserine, bifonazole, biphenamine, bromosalicylchloranilide, buclosamide, butoconazole, calcium propionate, candicidin, chlordantoin, chlormidazole, chlorphenesin, chlorquinaldol, cloconazole, cloxyquin, coparaffinate, m-cresyl acetate, cupric sulfate, dermostatin, diamthazole dihydrochloride, econazole, enilconazole, etisazol, exalamide, fenticonazole, filipin, flutrimazole, fungichromin, hachimycin, halethazole, hamycin, hexetidine, lanoconazole, loflucarban, lucensomycin, Magenta I, mepartricin, 2-(methoxymethyl)-5-nitrofuran, monensin, myxin, natamycin, neomycin undecylenate, nifuratel, oligomycins, omoconazole, ontianil, pecilocin, perimycin, pyrithione, pyrrolnitrin, rubijervine, salicylanilide, sertaconzole, siccanin, sulbentine, tenonitrozole, tolciclate, tolindate, triacetin, 2,4,6-tribromo-m-cresol, tubercidin, ujothion, undecylenic acid, viridin, and zinc propionate.

18. The method of claim 12, wherein the composition further comprises said at least one anti-microbial agent and said at least one anti-microbial agent is selected from the group consisting of cresol, clotrimazole, tolnaftate, terbinafine and tioconazole.

19. The method of claim 12, wherein the composition further comprises said at least one anti-microbial agent and said at least one anti-microbial agent is selected from the group consisting of benzoic acid and salts thereof, sorbic acid and salts thereof, propionic acid and salts thereof, boric acid and salts thereof, dehydroacetic acid, sulphurous acids, vanillic acids, phenol, cresol, chlorocresol, o-phenylphenol, chlorothymol, parabens, alkyl esters of parahydroxybenzoic acid, methyl-p-hydroxybenzoates, ethyl-p-hydroxybenzoates, propyl- p-hydroxybenzoates, benzyl-p-hydroxybenzoates and butyl-p-hydroxybenzoates, thimersal, phenylmercuric acetate and phenylmercuric nitrate, nitromersol, sodium ethylmercurithiosalicylate, benzalkonium chloride, cetylpyridinium chloride, benzethonium chloride, cetyltrimethyl ammonium bromide, chlorobutanol, phenoxy-2-ethanol, benzyl alcohol, β-phenylethyl alcohol, chlorohexidine, chloroform, 6-acetoxy-2,4-dimethyl-m-dioxane, 2,4,4′ trichloro-2′-hydroxy-diphenylether, imidizolidinyl urea compound, bromo-2nitropropanediol-1,3,5-bromo-5-nitrol-1,3 dioxane, 2-methyl-4-isothiazolin-3-one and 5 chloro derivative, and 1-(3-chloroallyl)-3,5,7-triazo 1-azoniaadamantane chloride.

20. The method of claim 12, wherein the composition further comprises said at least one therapeutic agent.

21. The method of claim 12, wherein the at least one anti-microbial or therapeutic agent is mixed with the polymerizable monomer composition immediately prior to applying the polymerizable monomer composition to the nasal area.

22. The method of claim 12, wherein the at least one anti-microbial or therapeutic agent is mixed with the polymerizable monomer composition during manufacture of the polymerizable monomer composition.

23. The method of claim 12, wherein the composition further comprises said at least one anti-microbial agent and said anti-microbial agent is a phenolic antioxidant.

24. The method of claim 23, wherein said antioxidant is a stabilizing agent for said monomer.

25. The method of claim 12, wherein the composition further comprises said at least one anti-microbial agent and said anti-microbial agent is butylparaben.

26. The method of claim 1, wherein said composition has a Sterility Assurance Level (SAL) of 10⁻³-10⁻⁶.

27. The method of claim 1, further comprising applying at least one of an anti-microbial or therapeutic agent to the nasal area before applying the adhesive composition.

28. The method of claim 27, wherein said method is for treating said epistaxis, and said nasal area is afflicted with said epistaxis.

29. The method of claim 27, wherein said method is for preventing said epistaxis, and said nasal area is susceptible to said epistaxis.

30. The method of claim 27, further comprising allowing the at least one applied anti-microbial or therapeutic agent to substantially dry before applying the adhesive composition.

31. The method of claim 27, wherein the anti-microbial agent is applied and is selected from the group consisting of parabens, cresols, and non-polymer-stabilized compounds.

32. The method of claim 27, wherein the anti-microbial agent is applied and is selected from the group consisting of alkyl parabens having an alkyl group of from 1-4 carbon atoms.

33. The method of claim 27, wherein the anti-microbial agent is applied and is selected from the group consisting of methylparaben, methylparaben sodium, ethylparaben, propylparaben, propylparaben sodium, butylparaben, cresol, chlorocresol, voriconazole, ketoconazole, fluconazole, itraconazole, miconazole, clotrimazole, saperconazole, neticonazole, oxiconazole, isoconazole, sulconazole, tercanazole, tioconazole, naftifine, SF86-327, nyastatin, amphotericin B, pimaricin, benzoic acid and salts thereof, sorbic acid and salts thereof, propionic acids and salts thereof, boric acid and salts thereof, dehydroacetic acid, sulphurous and vanillic acids, alkyl esters of pararhydrobenzoic acid, thimerosal, phenylmercuric borate, phenylmercuric acetate and phenylmercuric nitrate, nitromersol, sodium ethylmercurithiosalicylate, benzalkonium chloride, benzethonium chloride, benzoic acid, benzyl alcohol, cetylpyridinium chloride, chlorobutanol, dehydroacetic acid, o-phenylphenol, phenol, phenylethyl alcohol, potassium benzoate, potassium sorbate, sodium benzoate, sodium dehydroacetate, sodium propionate, sorbic acid, thimerosal, thymol, chlorothymol, alcohols, chlorobutanol, phenoxy-2-ethanol, benzyl alcohol, P-phenylethyl alcohol, chlorhexidine, 6-acetoxy-2,4-dimethyl-m-dioxane 2,4,4′ trichloro-2′-hydroxy-diphenylether, imidizoldinylether urea compound, bromo-2-nitropropanediol-1,3 5-bromo-5-nitrol-1,3 dioxane, 2-methyl 1-4-isothiazolin-3-one and 5 chloro derivative, 1-(3-chloroallyl)-3,5,7-triazo 1-azoniaadamantane chloride and formaldehyde, imidazolidinyl urea, morpholines, salicylic acids, benzoic acids, sodium iodides, potassium iodides, flucytosine, 5-flucytosine, griseofulvin, terbinafine, cidofovir, famicoclovir, valacyclovir, echinocandins, pneumocandins, pradimicins, benanomicins, nikkomycins, amorolfine, polyoxins, duanorubicin citrate, doxorubicin hydrochloride, tolnaftate, ciclopirox, butenafine, ergestrol biosynthesis inhibitors, acrisorein, 3-amino-4-hydroxybutyric acid, ammonium mercuric chloride, amorolfine, anthralin, azaserine, bifonazole, biphenamine, bromosalicylchloranilide, buclosamide, butoconazole, calcium propionate, candicidin, chlordantoin, chlormidazole, chlorphenesin, chlorquinaldol, cloconazole, cloxyquin, coparaffinate, m-cresyl acetate, cupric sulfate, dermostatin, diamthazole dihydrochloride, econazole, enilconazole, etisazol, exalamide, fenticonazole, filipin, flutrimazole, fungichromin, hachimycin, halethazole, hamycin, hexetidine, lanoconazole, loflucarban, lucensomycin, Magenta I, mepartricin, 2-(methoxymethyl)-5-nitrofuran, monensin, myxin, natamycin, neomycin undecylenate, nifuratel, oligomycins, omoconazole, ontianil, pecilocin, perimycin, pyrithione, pyrrolnitrin, rubijervine, salicylanilide, sertaconzole, siccanin, sulbentine, tenonitrozole, tolciclate, tolindate, triacetin, 2,4,6-tribromo-m-cresol, tubercidin, ujothion, undecylenic acid, viridin, and zinc propionate.

34. The method of claim 27, wherein the anti-microbial agent is applied and is selected from the group consisting of elemental metals and metal compounds.

35. The method of claim 27, wherein the anti-microbial agent is applied and further comprises at least one of a diluent and a carrier.

36. The method of claim 27, wherein the therapeutic agent is applied.

37. The method of claim 1, wherein said adhesive composition is applied directly to said nasal area, and said adhesive composition does not include an anti-microbial or therapeutic agent.

38. The method of claim 37, wherein said polymer film has anti-microbial effects at said nasal area.

39. A method of treating or preventing epistaxis, the method comprising the steps of:
a. applying at least one of an antimicrobial or therapeutic agent to a nasal area that is afflicted with or susceptible to epistaxis;
b. applying a polymerizable monomer composition to said nasal area over the at least one applied anti-microbial or therapeutic agent, wherein said composition comprises a 1,1-disubstituted ethylene monomer; and
c. allowing said polymerizable monomer composition to polymerize to form a polymer film over said nasal area and said at least one anti-microbial or therapeutic agent.

40. The method of claim 39, wherein said method is for treating said epistaxis, and said nasal area is afflicted with said epistaxis.

41. The method of claim 39, wherein said method is for preventing said epistaxis, and said nasal area is susceptible to said epistaxis.

42. A method of treating or preventing epistaxis, the method comprising:
a. combining a synthetic or semi-synthetic polymerizable monomer polymerizable monomer composition and at least one of an anti-microbial or therapeutic agent to form a mixture;
b. applying said mixture to a nasal area that is afflicted with or susceptible to epistaxis; and
c. allowing said mixture to polymerize to form a polymer film over said nasal area.

43. The method of claim 42, wherein said method is for treating said epistaxis, and said nasal area is afflicted with said epistaxis.

44. The method of claim 42, wherein said method is for preventing said epistaxis, and said nasal area is susceptible to said epistaxis.

45. A composition for treating or preventing epistaxis, comprising:
a synthetic or semi-synthetic polymerizable monomer, and
a vasoconstrictor.

46. The composition of claim 45, wherein said vasoconstrictor is selected from the group consisting of Aesculus hippocastanum (Hippocastanaceae), Corylus avellana (Betulaceae), Ephedra sinica (Ma Huang), Hamamelis virginiana (Witch Hazel), Hydrastis canadensis (Goldenseal), Lycopus virginicus (Bugleweed), Aspidosperma quebracho (Quebracho blanco), Cupressus sempervirens (Cupressaceae), Cytisus scoparius (Fabaceae), Gossypium arboreum (Malvaceae), Gossypium herbaceum (Malvaceae), Hedera helix (Araliaceae), Phellodendron amurense (Rutaceae), Plectranthus mollis (Lamiaceae), Polygonum hydropiper (Polygonaceae), Seseli sibiricum (Apiaceae), Strychnos ignatius (Loganiaceae), Strychnos nux-vomica (Loganiaceae), Urtica dioica (Urticaceae), phenylephrine hydrochloride, etilefrine hydrochloride, acetylcholine, bradykinin, naphazoline hydrochloride, Angiotensin II (AII), epinephrine, and cocaine.
